(19) United States
(12) Patent Application Publication
(10) Pub. No.: US 2008/0275464 A1
(43) Pub. Date: Nov. 6, 2008

(54) ARTICULATING RETRIEVAL DEVICE

(75) Inventors: Robert M. Abrams, Los Gatos, CA (US); Riten Parikh, San Jose, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/173,356

(22) Filed: Jul. 15, 2008

Related U.S. Application Data
(62) Division of application No. 11/092,644, filed on Mar. 29, 2005.

(51) Int. Cl. A61B 17/26

(52) U.S. Cl. 606/113

(57) ABSTRACT

Device and methods for removing a foreign object from a body lumen are disclosed. A retrieval device in accordance with an exemplary embodiment of the present invention may include an elongated member including a flexible collector element, and a core wire that can be engaged by the physician to actuate the collector element between a first position and a second position with the body. The collector element may comprise a coiled section including a coiled flat ribbon adapted to assume a substantially straight shape in the first position and an expanded shape forming one or more helically oriented loops in the second position. The collector element may also comprise one or more fibers operatively coupled to the coiled section.

### RELATED APPLICATIONS

This application is a divisional application of U.S. application Ser. No. 11/092,644 filed Mar. 29, 2005.

### FIELD

The present invention relates generally to the field of medical devices. More specifically, the present invention pertains to devices for removing foreign objects within a body lumen.

### BACKGROUND

Embolectomy devices such as inflatable catheters and clot pullers are used in a variety of applications to remove blood clots or other foreign objects from a blood vessel. In applications involving the cerebrovasculature, for example, such devices may be used to remove a blood clot from an intracranial artery for the treatment of ischemic stroke. The formation of thrombus within the artery may partially block or totally occlude the flow of blood through the artery, preventing blood from reaching the brain or other vital organs. Such thrombolytic events may also be exacerbated by atherosclerosis, a vascular disease that causes the vessels to become tortuous and narrowed. The tortuosity or narrowness of the vessel may, in certain circumstances, lead to the formation of atherosclerotic plaque, which can cause further complications to the body if not treated.

In embolectomy procedures for removing blood clots, a delivery catheter or sheath is typically inserted percutaneously into the body (e.g. via the femoral, jugular or antecubital veins) and advanced to a target site within the body containing the clot. In some applications, for example, a Fogarty catheter or other such delivery device can be used to transport the embolectomy device in a collapsed position to the site of the clot. To ascertain the precise location of the clot within the vessel, a radiopaque die can be injected into the body to permit the occluded vessel to be radiographically visualized with the aid of a fluoroscope. Once positioned, the embolectomy device is then deployed out from within the delivery device, causing the embolectomy device to expand in the vessel. The embolectomy device can then be manipulated within the vessel to remove the clot from the vessel wall, if necessary. A wire basket, coil, membrane or other collector element can be used to capture the clot as it is dislodged from the vessel wall. Once captured, the embolectomy device is then loaded into a retrieval catheter and withdrawn from the patient's body.

The ability of many embolectomy devices to capture blood clots or other foreign objects may be limited by the ability of the collector element to expand and positively engage the blood clot surface. In those embodiments employing an articulating wire coil, for example, the efficacy of the device to ensnare the foreign object may be limited by the ability of the wire coil to adequately expand about the surface of the object. In some cases, the shape of the coil turns may affect the ability of the embolectomy device to dislodge and grip the blot clot. Other factors such as the mechanical strength and/or size of the collector element may also reduce the effectiveness of the device in capturing blood clots in certain applications.

### SUMMARY

The present invention pertains to devices for removing foreign objects within a body lumen. A retrieval device in accordance with an exemplary embodiment of the present invention can include an elongated member having a flexible coil section actuatable between a collapsed shape and an expanded shape within the body. The coil section can include a coiled flat ribbon that, when expanded using a core wire operatively coupled to an optional actuation mechanism, causes the coiled flat ribbon to assume an expanded shape having one or more helically oriented loops. A distal section of the core wire can be configured to yield under tension at a force lower than that of a proximal section thereof, causing the coil section to articulate when a tensile force is applied to the core wire. A textured surface formed on one or more of the coil turns can be used in certain embodiments to facilitate gripping of the blood clot as the retrieval device is manipulated within the blood vessel.

The size and number of loops can be varied to permit the retrieval device to be utilized in a variety of applications, as desired. In some embodiments, the expanded loops may have a distally tapering shape with a closed configuration at one end that prevents the blood clot from slipping through the structure as the retrieval device is engaged proximally within the blood vessel, or when the device is loaded within the interior of a retrieval catheter. In certain embodiments, a number of polymer fibers can be attached to various locations of the coil section to limit the amount of longitudinal stretching that occurs to the coil section as the retrieval device is engaged within the body. In some applications, the polymer fibers also function by increasing the total surface area of the retrieval device.

In another illustrative embodiment, the retrieval device can include a pusher wire, a filter basket operatively coupled to the pusher wire and including a plurality of filter struts that form a number of expandable basket cells for capturing the blood clot, and a core wire operatively coupled to one or more of the filter struts. The filter basket can be configured to expand from a collapsed position to an expanded position in response to a tensile force applied to the core wire, allowing the structure to assume a relatively low profile within a delivery catheter or sheath.

### BRIEF DESCRIPTION OF THE DRAWINGS

FIG. 1 is perspective view showing a retrieval device in accordance with an exemplary embodiment of the present invention;

FIG. 2 is a perspective view showing the illustrative retrieval device of FIG. 1 in a second position;

FIG. 3 is a cross-sectional view showing the distal coil section of FIG. 1 in greater detail;

FIG. 4 is an expanded view showing the coil turns of FIG. 3 having a textured surface;

FIG. 5 is a cross-sectional view showing the distal coil section of FIG. 1 in a second position;

FIG. 6 is a partial cross-sectional view showing the retrieval device of FIG. 1 advanced to a target site within a blood vessel;

FIG. 7 is a partial cross-sectional view showing the retrieval device of FIG. 1 in a second position engaged along the wall of the blood vessel;

FIG. 8 is a partial cross-sectional view showing the retrieval device of FIG. 1 in a third position collapsed about the blood clot;

FIG. 9 is a partial cross-sectional view showing the retrieval device of FIG. 1 in a fourth position loaded into a catheter;

FIG. 10 is a perspective view showing the distal portion of a retrieval device in accordance with another exemplary embodiment of the present invention;

FIG. 11 is a perspective view showing the distal portion of a retrieval device in accordance with another exemplary embodiment of the present invention;

FIG. 12 is a perspective view showing the distal portion of a retrieval device in accordance with another exemplary embodiment of the present invention;

FIG. 13 is a top view of the filter basket of FIG. 12, showing the filter basket prior to assembly on the pusher wire; and

FIG. 14 is another top view of the filter basket of FIG. 12, showing the filter basket with a polymeric web covering.

### DETAILED DESCRIPTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

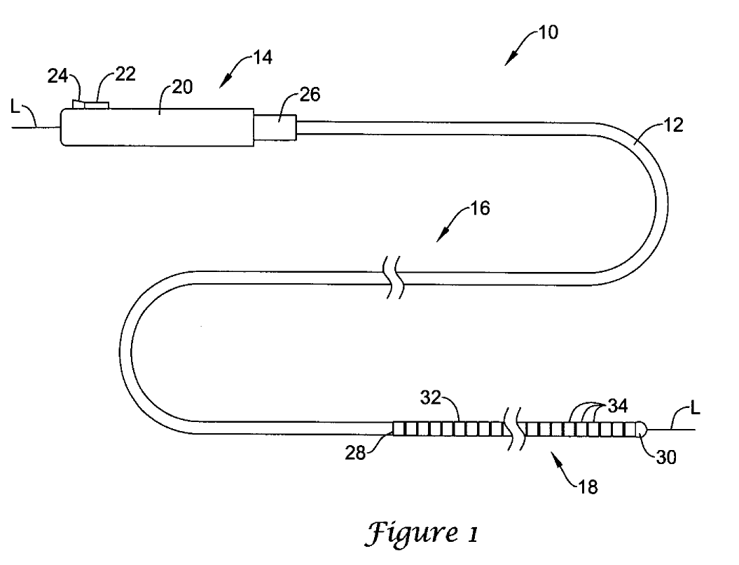

FIG. 1 is perspective view showing a retrieval device 10 in accordance with an exemplary embodiment of the present invention. As shown in a first (i.e. collapsed) position in FIG. 1, the retrieval device 10 can include an elongated member 12 having a proximal section 14, a longitudinally extending support body 16, and a distal coil section 18. As is described in greater detail below, the retrieval device 10 can be actuated between a collapsed position wherein the distal coil section 18 assumes a substantially straight shape having a relatively low profile for transport of the retrieval device 10 through the vasculature, and an expanded position wherein the distal coil section 18 articulates in the general shape of a helix for removal of a blood clot within the body.

The proximal section 14 of the elongated member 12 can include a handle 20 that can be used by the physician to manipulate the retrieval device 10 from a position outside of the patient's body. The handle 20 may include a slidable thumbpiece actuator 22 that can be engaged by the physician's thumb between a first (i.e. retracted) position and a second (i.e. forward) position to actuate the retrieval device 10 between the collapsed and expanded positions. The thumbpiece actuator 22 can be configured to slide back and forth within a slot disposed along the length of the handle 20, allowing the physician to actuate the retrieval device 10 by moving the thumbpiece actuator 22 forward with the thumb while gripping the handle 20. In certain embodiments, the retrieval device 10 may include an internal spring mechanism that can be used to releasably lock the thumbpiece actuator 22 in position within the slot. A button 24 or other suitable mechanism can be provided to subsequently release the thumbpiece actuator 22 within the slot, allowing the physician to reposition the thumbpiece actuator 22 to another position, if desired.

The support body 16 of the elongated member 12 can have a tubular construction adapted to transmit axial and rotational forces exerted on the handle 20 to the distal coil section 18. In contrast to the flexible distal coil section 18, the support body 16 may have a relatively stiff construction with sufficient column strength and rigidity to withstand buckling or bulging as the retrieval device 10 is engaged within the patient's body. The wall thickness of the support body 16 may be generally uniform along its length, or may vary along its length to alter the flexibility or bending characteristics of the retrieval device 10, as desired. A strain relief 26 can be provided in certain embodiments to reduce stress buildup at the transition between the proximal section 14 and the support body 16. While the illustrative support body 16 depicted in FIG. 1 is formed from a substantially solid tubular structure, it should be understood that other suitable structures such as a spring coil or braid could be employed.

The materials used in forming the support body 16 can be selected to impart a desired mechanical characteristic to the retrieval device 10. Typically, the support body 16 will be formed of a material or materials having a sufficient stiffness or rigidity to permit the retrieval device 10 to be manipulated within the patient's body without buckling or bulging. Examples of suitable materials that can be used in forming the support body 16 may include, but are not limited to, metals such as stainless steel (e.g. 304V, 316L, etc.), polymers such as polyether block amide (PEBA), polyethylene terapthalate (PET), polytetrafluoroethylene (PTFE), or metal-polymer composites such as stainless steel reinforced hypotube. In certain embodiments, a superelastic material such as nickel-titanium alloy (Nitinol) can be utilized, allowing the retrieval device 10 to undergo significant bending or flexion within the body without imparting a residual strain to the material.

The distal coil section 18 of the retrieval device 10 may have a proximal end 28 and a distal end 30. At the proximal end 28 of the distal coil section 18, the elongated member 12 may transition from the distal end of the support body 16 to a flexible wire coil 32 having a number of individual coil turns 34 that can be articulated in a path away from the general longitudinal axis L of the retrieval device 10. The distal end 30 of the distal coil section 18 may have a rounded or bulbous shape to reduce trauma to the vessel wall as the retrieval device 10 is traversed through the vasculature.

To permit visualization within the body, at least a portion of the distal coil section 18 can be loaded with or otherwise formed of a radiopaque material. Examples of suitable radiopaque materials can include, but are not limited to, gold (Ag), iridium (Ir), platinum (Pt), silver (Au), tantalum (Ta), tungsten (W), bismuth subcarbonate ((BiO)₂CO₃), and barium sulfate (BaSO₄). In certain embodiments, the distal coil section 18 can be made of a coilable metal, polymer, or metal-polymer material, and then coated with a radiopaque layer or coating to enhance radiopacity. In addition, and in some embodiments, radiopaque marker bands can be placed on one or more of the coil turns 34, if desired.

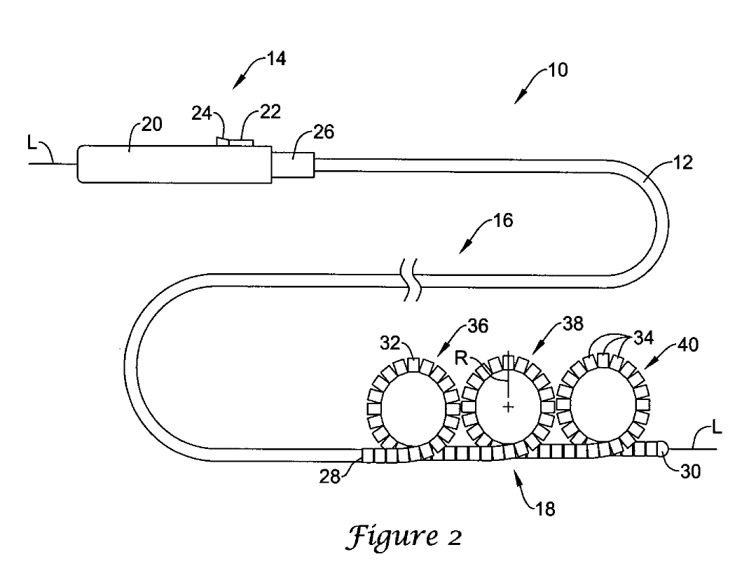

FIG. 2 is a perspective view showing the illustrative retrieval device 10 of FIG. 1 in a second (i.e. expanded) position. As can be seen in FIG. 2, the distal coil section 18 can be configured to articulate into an expanded position in response to forward movement of the thumbpiece actuator 22 within the handle 20. In an expanded position, the coil turns 32 can be configured to bend and orient to a pre-defined (i.e. equilibrium) helical shape, forming a number of helically oriented loops that align circumferentially with the inner wall of the blood vessel.

In the illustrative embodiment depicted in FIG. 2, for example, the distal coil section 18 is shown having three individual loops 36,38,40 in the expanded position, each loop 36,38,40 having a radius R similar to the radius of the blood vessel in which the retrieval device 10 is to be inserted into. The distal coil section 18 can have a greater or lesser number of loops than that depicted in FIG. 2, however, depending on the particular application, the size of the blood vessel, the size of the blood clot, as well as other factors. If, for example, the blood clot to be excised from the vessel wall is relatively long, or is located at the juncture of multiple lumens, a retrieval device having a greater number of loops can be employed. Conversely, if the blood clot to be excised from the vessel wall is relatively short, or is located in a vessel having a relatively short length, a retrieval device having a lesser number of loops can be employed.

The size and shape of the loops 36,38,40 can be further customized to treat any number of pathologies and/or to facilitate insertion of the retrieval device 10 in hard-to-reach regions of the vasculature (e.g. at a bifurcation branch). Typically, the loops 36,38,40 will be selected to expand to a size that encloses a volume slightly larger than the anticipated volume of the blood clot, although other sizes may be desired in certain applications. Collectively, the loops 36,38,40 may define an interior space that receives the incoming blood clot as it is dislodged from the vessel wall.

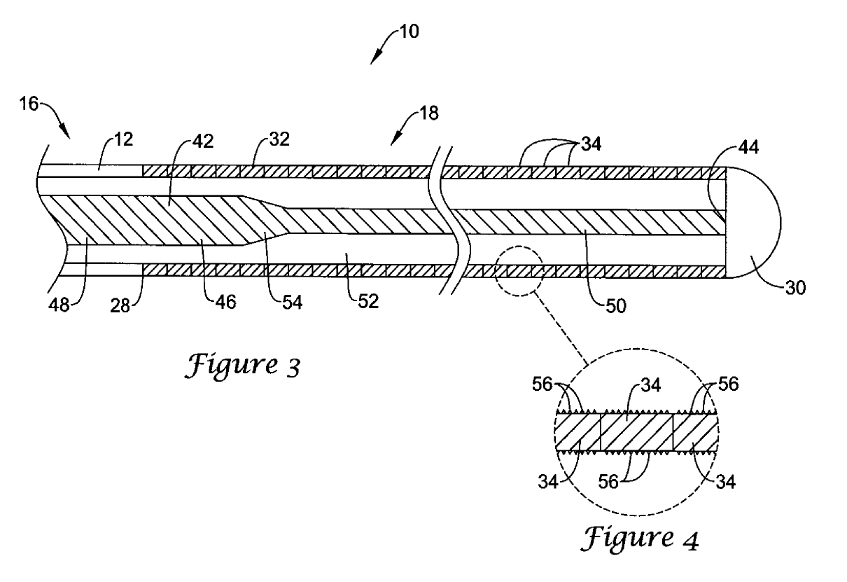

FIG. 3 is a cross-sectional view showing the distal coil section 18 of FIG. 1 in greater detail. As shown in FIG. 3, the retrieval device 10 may further include a core wire 42 operatively coupled at a proximal end (not shown) to the thumbpiece actuator 22, and at a distal end 44 thereof to the distal end 30 of the distal coil section 18. The core wire 42 may have a proximal section 46 extending through an interior lumen 48 of support body 16, and a distal section 50 that extends through an interior lumen 52 of the distal coil section 18.

The distal section 50 of the core wire 42 can be configured to yield under tension at a force lower than that of the proximal section 48, causing the distal section 50 to displace and assume a coiled shape when the core wire 42 is advanced distally using the thumbpiece actuator 22. The distal section 50 can be configured to displace only when a certain threshold tensile force is applied to the core wire 42, at which point the core wire 42 material readily responds to each addition unit of force applied thereto by displacing into the coiled state.

The ability of the distal section 50 of the core wire 42 to yield at a rate greater than the proximal section 48 thereof can be accomplished by altering the cross-sectional area of each section 48,50. In the illustrative embodiment of FIG. 3, for example, the distal section 50 of the core wire 42 may have a transverse cross-sectional area that is smaller than that of the proximal section 48, imparting greater bendability and flexibility to the distal section 50. A tapered region 54 of the core wire 42 located at the juncture of the proximal and distal sections 48,50 can be configured to gradually transition the profile of the core wire 42. In other embodiments, the core wire 42 may continuously change in cross-section along its length, or, alternatively, may transition in cross-section at multiple regions along its length, if desired.

The materials used in forming the proximal and distal sections 48,50 can be further selected to permit the distal section 50 of the core wire 42 to yield under tension at a rate greater than the proximal section thereof 48. In certain embodiments, for example, the proximal section 48 may be formed from a stiff or rigid material having a relatively high modulus of elasticity, whereas the distal section 50 may be formed from a bendable or flexible material having a relatively low modulus of elasticity that is capable of bending appreciably in response to the same applied stress. By way of example and not limitation, the proximal section 48 may comprise a relatively stiff material such as stainless steel whereas the distal section 50 may comprise a relatively flexible, superelastic material such as nickel-titanium alloy (Nitinol). In such case, the proximal and distal sections 48,50 of the core wire 42 could have the same cross-sectional area while still exhibiting the desired yielding characteristics, as described above.

The types of material or materials used in forming the proximal and distal sections 48,50 of the core wire 42 will typically depend on the desired mechanical characteristics of the retrieval device 10, the materials used in fabricating the support body 16 and distal coil section 18, the size and shape of the coil turns 34, as well as other factors. In those embodiments wherein the distal section 50 comprises a superelastic material, a desired shape can be imparted to the core wire 42 by heating the material beyond its final austenitic temperature A_{f}, and then bending the material to a desired shape. Once cooled, and when subjected to further deformation during use, the distal section 50 can be configured to revert to its heat-induced (i.e. coiled) state.

As can be further seen in FIG. 3, each of the coil turns 34 may be formed from a coiled flat ribbon having a rectangular transverse cross-sectional area. The coiled flat ribbon may have either a smooth surface or a textured surface depending on the amount the amount of force necessary to excise the blood clot from the vessel wall, the amount of gripping and/or tackiness required to positively engage the blood clot, as well as other factors. In use, the edges of the coil turns 34 act to positively engage the surface of the blood clot, improving the ability of the coil turns 34 to mechanically grip the blood clot as the retrieval device 10 is manipulated within the blood vessel. The coil turns 34 may be tightly wound together, as shown, or may be loosely wound to impart greater flexibility to the distal coil section 18, as desired. Other factors such as the pitch and the number of the coil turns 34 can be selected to accommodate blood clots of different size, or to permit the retrieval device 10 to be inserted into variously sized vessels of the body. In some embodiments, the coil turns 34 of the coiled flat ribbon can formed by helically wrapping a flat piece of ribbon about a mandrel, and then applying heat to the material to set the desired shape. While the illustrative coil turns 34 are shown having a rectangular transverse cross-sectional area in FIG. 3, it should be understood that the coil turns 34 may assume other shapes (e.g. circular, oval, triangular, etc.), as desired.

One or more of the coil turns 34 may have a textured surface that can be further utilized to grip the blood clot as the retrieval device 10 is manipulated within the blood vessel. As shown in greater detail in FIG. 4, for example, a number of bumps or protrusions 36 formed on the edges and/or sides of the coil turns 34 can be provided to facilitate gripping of the coil turns 34 to the blood clot surface. The textured surface can be formed by applying a metal or polymer nanoporous coating to the surface of each coil turn 34 by sputter deposition, electroplating, epitaxial growth, or other suitable technique. A nanoporous coating, as used herein, is understood to be a material having a pore size in the range of about 1 nm to 500 nm, and more specifically, 1 nm to 200 nm. In use, the nanoporous coating provides an open cell surface that enhances the ability of the retrieval device 10 to grip the blood clot by increasing the overall surface area of the coil turns 34. The nanoporous further provides additional tackiness that facilitates adherence of the blood clot to the coil turns 32 once contacted therewith.

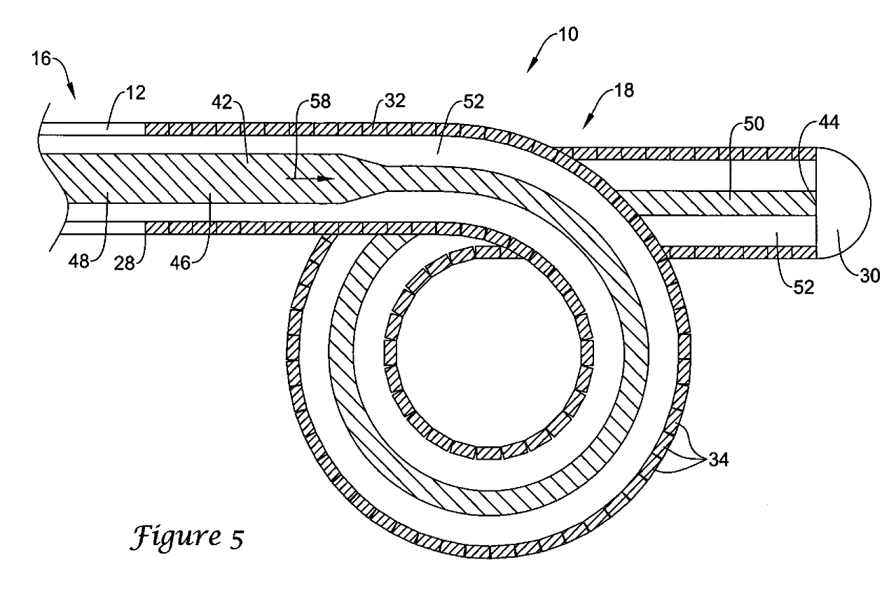

FIG. 5 is a cross-sectional view showing the distal coil section 18 of FIG. 1 in a second (i.e. coiled) position. As indicated generally by arrow 58 in FIG. 5, advancement of the core wire 42 in the distal direction relative to the elongated member 12 increases the tensile force exerted on the distal coil section 18, inducing stress at each point along the length of the core wire 42. Because the distal section 50 of the core wire 42 has a smaller cross-sectional area than the proximal section 48, the stress induced within the distal section 50 is greater than that experienced by the proximal section 48. This increase in stress within the distal section 50 causes the distal section 50 to undergo a greater strain than at the proximal section 48, thus becoming significantly longer in length. A similar effect occurs in those embodiments wherein the distal section comprises a material having a modulus of elasticity smaller than the proximal section 48 thereof. The increased amount of strain induced in the distal section 50 from either the decrease in cross-sectional area and/or the selection of certain types of materials causes the distal coil section 18 to revert to its equilibrium coiled state, as shown in FIG. 5.

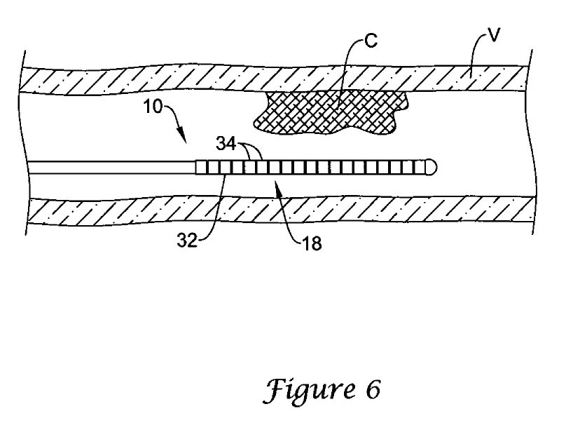

Referring now to FIGS. 6-9, an illustrative method of retrieving a foreign object within a blood vessel will now be described with respect to the illustrative retrieval device 10 of FIG. 1. In preparation for insertion within the body, and if necessary, the thumbpiece actuator 22 can be retracted proximally, causing the core wire 42 to release the tension on the distal coil section 18 and allowing the coil turns 34 to assume their low profile (i.e. collapsed) position. In a collapsed position, the physician may insert the retrieval device 10 percutaneously into the body and advance the device 10 through the vasculature to a desired location adjacent a blood clot C, as shown in FIG. 6. If desired, a guide catheter or other suitable guiding instrument may be utilized to help guide the retrieval device 10 within the body.

Once positioned at the site of the blood clot C, the distal coil section 18 of the retrieval device 10 can then be actuated within the blood vessel V, causing the coil turns 34 to expand and assume their coiled state. Actuation of the distal coil section 18 may be accomplished, for example, by sliding the thumbpiece actuator 22 forward within the handle 20 (see FIG. 2), causing the core wire 42 to tension and strain, thereby permitting the coil turns 34 to revert to their coiled position.

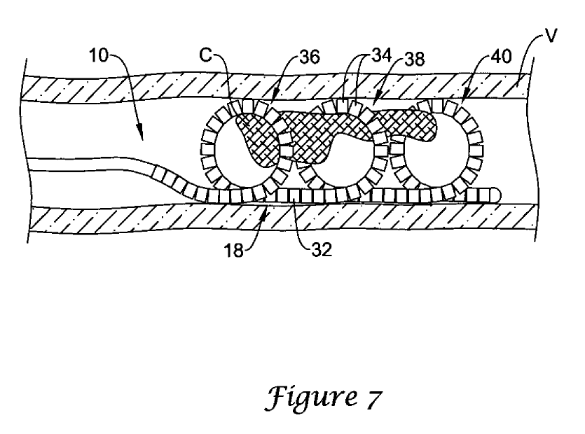

With the distal coil section 18 expanded within the blood vessel V, the physician can then manipulate the retrieval device 10 to excise the blood clot C from the inner wall of the blood vessel V, as shown in a second position in FIG. 7. In certain techniques, for example, removal of the blood clot C from the wall of the blood vessel V may be accomplished by positioning one or more of the expanded loops 36,38,40 distally of the blood clot C, and then pulling the elongated member 12 proximally a distance to dislodge the blood clot C from the vessel wall. The engagement of the distal coil section 18 against the wall of the blood vessel V in this manner acts to shear the blood clot C from the vessel wall, forcing it into the interior space defined by the loops 36,38,40.

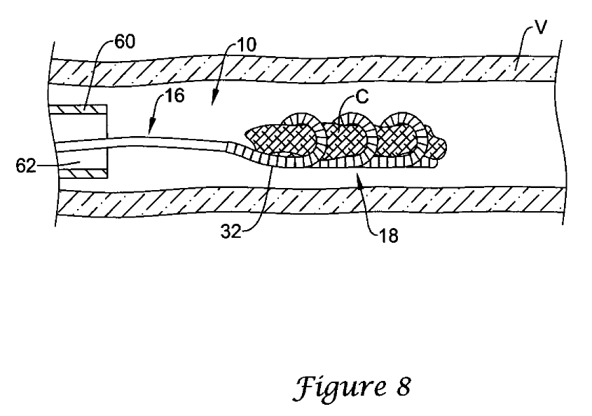

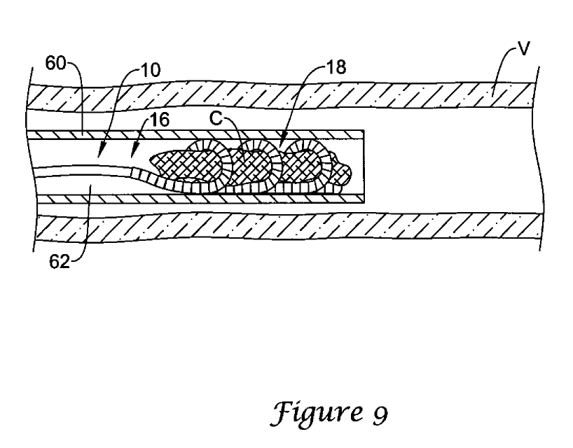

Once the blood clot C has been excised from the vessel wall, the physician may then retract the thumbpiece actuator 22 proximally within the handle 20, causing the distal coil section 18 to revert to its collapsed position, as shown in a third position in FIG. 8. As shown in FIG. 8, a catheter 60 having an interior lumen 62 adapted to receive the collapsed retrieval device 10 and captured blood clot C can then be inserted into the body and advanced to the target site. Once positioned at the target site, the retrieval device 10 can then be loaded into the interior lumen 64, as shown in a fourth position in FIG. 9. Loading of the retrieval device 10 into the interior lumen 62 can be accomplished by withdrawing the retrieval device 10 proximally while holding the catheter 60 stationary within the blood vessel V, or, alternatively, by holding the retrieval device 10 stationary within the blood vessel V while advancing the catheter 60 distally. Once loaded, the catheter 60 and accompanying retrieval device 10 can then be removed from the body.

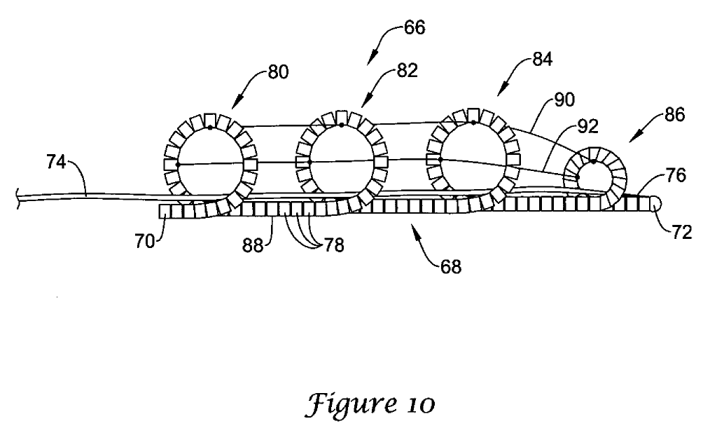

FIG. 10 is a perspective view showing the distal portion of a retrieval device 66 in accordance with another exemplary embodiment of the present invention. As shown in FIG. 10, the retrieval device 66 can include a coil section 68 having a proximal end 70 and a distal end 72. In the illustrative embodiment of FIG. 10, the proximal end 72 of the retrieval device 70 can be connected directly to a core wire 74 having a proximal end (not shown) and a distal end 76. The distal end 72 of the coil section 68 can be connected to the distal end 76 of the core wire 74, and can have a rounded or bulbous shape to reduce trauma to the vessel wall as the retrieval device 66 is manipulated within the body. In some embodiments, the coil section 68 can be loaded with or otherwise formed of a radiopaque material, and/or can include radiopaque marker bands on one or more of its coil turns 78, if desired.

The coil section 68 of the retrieval device 66 can be configured to articulate from a collapsed position to an expanded position in response to axial movement of the core wire 74 by the physician. In an expanded position depicted in FIG. 10, the coil turns 78 can be configured to bend and orient to a pre-defined helical shape, forming a number of helically oriented loops 80,82,84,86 that align circumferentially with the inner wall of the blood vessel. The loops 80,82,84,86 can each be configured to radially expand the same amount within the blood vessel, or can radially expand by varying amounts depending on the application. In the illustrative embodiment of FIG. 10, for example, the distal-most loop 86 is shown having a smaller radius than that of the other loops 80,82,84. In use, the smaller radius on the distal-most loop 86 acts to close-off the distal portion of the coil section 68 to prevent the blood clot from slipping through the structure as the retrieval device 10 is manipulated proximally within the blood vessel, or when the device 10 is loaded into a retrieval catheter.

The coil turns 78 may be formed from a coiled flat ribbon having a rectangular cross-sectional area, or can comprise some other cross-sectional shape, as desired. In some embodiments, one or more of the coil turns 78 may have a textured surface 88 thereon, which as described above, can be formed by applying a metal or polymer nanoporous coating to the surface of each coil turn 78. Alternatively, and in other embodiments, the coil turns 78 may have a relatively smooth surface 88.

Actuation of the coil section 68 between the collapsed position and the expanded position can be accomplished by pulling the core wire 74 proximally, releasing the tension provided on the distal end 76 by the core wire 74 and allowing the coil turns 78 to assume their equilibrium coiled shape, as shown. A number of polymer fibers 90,92 attached to various locations of the coil section 68 can be provided to limit the amount of longitudinal stretching that occurs to the coil section 68 as the retrieval device 66 is engaged within the body. The polymer fibers also function by increasing the total surface area of the retrieval device 10.

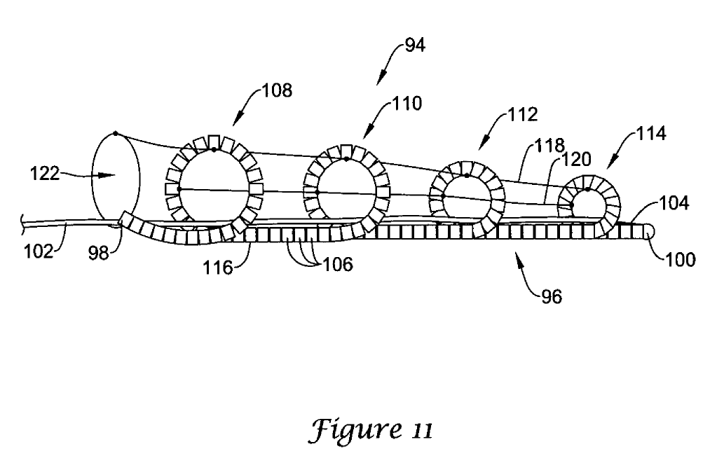

FIG. 11 is a perspective view showing the distal portion of a retrieval device 94 in accordance with another exemplary embodiment of the present invention. As shown in FIG. 11, the retrieval device 94 can include a coil section 96 having a proximal end 98 and a distal end 100. As with the embodiment of FIG. 10, the proximal end 98 of the retrieval device 94 can be connected directly to a core wire 102 having a proximal end (not shown) and a distal end 104. The distal end 100 of the coil section 96 can be connected to the distal end 104 of the core wire 102, and can have a rounded or bulbous shape to reduce trauma to the vessel wall as the retrieval device 94 is manipulated within the body. As with other embodiments herein, the coil section 96 can be loaded with or otherwise formed of a radiopaque material, and/or can include radiopaque marker bands on one or more of its coil turns 106, if desired.

The coil section 96 of the retrieval device 94 can be configured to articulate from a collapsed position to an expanded position in a manner similar to that described above with respect to FIG. 10. In the illustrative embodiment of FIG. 11, however, the expanded loops 108,110,112,114 may have a tapered shape wherein each successive loop in the distal direction 108,110,112,114 is reduced in size. Such reduction in size of the loops 108,110,112,114 in the distal direction acts to close-off the distal portion of the coil section 96 to prevent the blood clot from slipping through the structure as the retrieval device 94 is manipulated proximally within the blood vessel, or when the device 94 is loaded into a retrieval catheter and/or guide catheter.

The coil turns 106 can be formed from a coiled flat ribbon having a rectangular cross-sectional area, or can comprise some other cross-sectional shape, as desired. In some embodiments, one or more of the coil turns 106 may have a textured surface 116 thereon, which as described above, can be formed by applying a metal or polymer nanoporous coating to the surface of each coil turn 106.

Actuation of the coil section 96 between the collapsed position and the expanded position can be accomplished in a manner similar to that described above with respect to FIG. 10, by pulling the core wire 102 proximally. A number of polymer fibers 118,120 attached to various locations of the coil section 96 can be provided to limit the amount of longitudinal stretching that occurs to the coil section 96 as the retrieval device 94 is engaged within the body. In certain embodiments, a portion of the polymer fiber 118 located furthest away from the core wire 102 may extend a distance proximally of the proximal-most loop 108, and can be looped around to form a mouth 122 of the retrieval device 94.

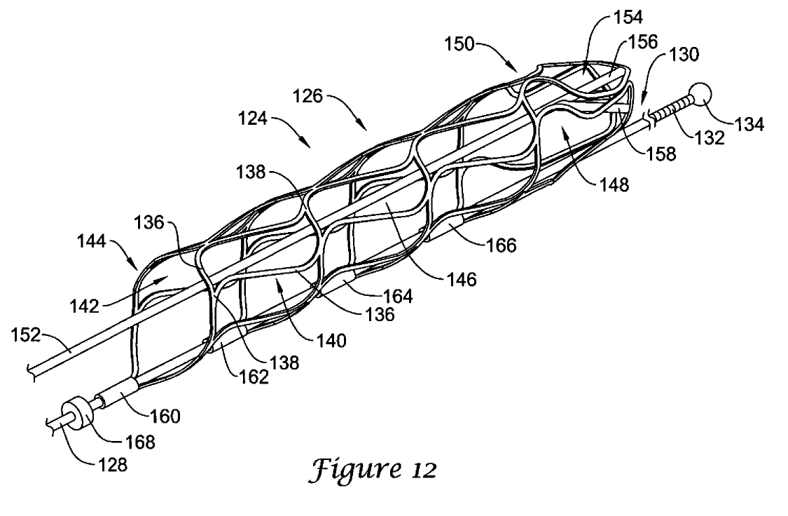

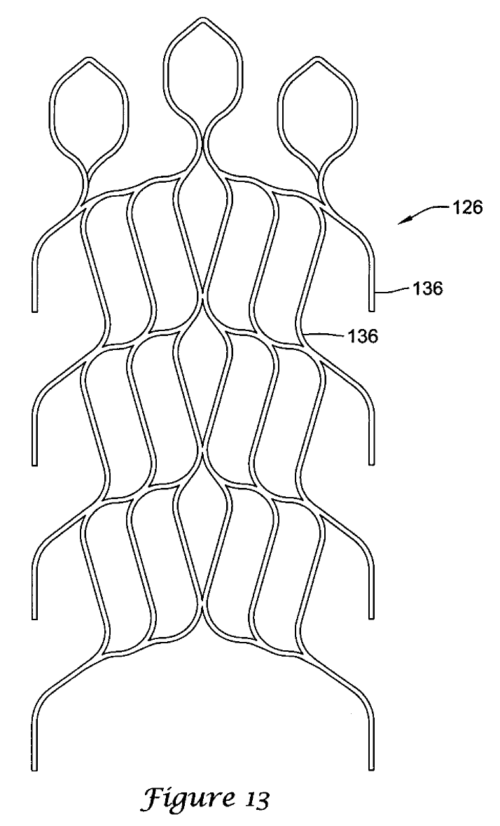

FIG. 12 is a perspective view showing the distal portion of a retrieval device 124 in accordance with another exemplary embodiment of the present invention. As shown in FIG. 12, the retrieval device 124 can include a filter basket 126 operatively coupled to a pusher wire 128 that can be manipulated by the physician from a position outside of the patient's body to engage the retrieval device 124 within a blood vessel. The pusher wire 128 can have a proximal section (not shown) adapted to lie outside of the patient's body, and a distal section 130 adapted to support the filter basket 126 within a blood vessel. The pusher wire 128 can be configured similar to other guiding members used in the art (e.g. guidewires), having the ability to transmit axial and rotational motion from the proximal section of the pusher wire 128 to the distal end 130 thereof. A radiopaque spring coil 132 disposed about the distal section 130 may provide additional stiffness to the pusher wire 128 while providing a visual reference point when used in conjunction with a fluoroscope. An atraumatic distal tip 134 having a rounded or bulbous shape may also be employed to reduce trauma to the body, if desired.

The filter basket 126 can include several filter struts 136 and connecting junctures 138 forming a number of basket cells 140 adapted to radially surround and capture the blood clot therein. The filter basket 126 can include an opening 142 in a proximal section 144 thereof, which receives the incoming blood clot as it is dislodged from the vessel wall. The basket cells 140 located on the proximal section 144 of the filter basket 126 can be arranged in a circumferential manner, forming an inner lumen 146 that receives the incoming blood clot. Several basket cells 148 located at a distal section 150 of the filter basket 126 can have a closed configuration, preventing the blood clot or other emboli from escaping the filter basket 126 once captured therein. The profile of the filter basket 126 can be generally cylindrical, conical, or other desired shape.

The filter struts 136 forming the basket cells 140 can be made flexible to permit the filter basket 126 to move and expand in multiple directions, including both radially and longitudinally within the blood vessel. In certain embodiments, the filter struts 136 may comprise a superelastic and/or shape memory material such as nickel-titanium alloy (Nitinol), allowing the filter struts 136 to bend and flex significantly without permanently deforming. Other suitable metals, polymers, or metal-polymer composites may be employed, however, depending on the application.

A core wire 152 extending through the inner lumen 146 of the filter basket 126 can be used to actuate the filter basket 126 between a collapsed position and an expanded position within the body. The core wire 152 may have a proximal section (not shown) that can be manipulated by the physician at a location outside of the patient's body, and a distal section 154 that is attached to the closed basket cells 148 located at the distal section 150 of the filter basket 126. The distal section 154 of the core wire 152 can be connected to each of the closed basket cells 148 via a number of wire segments 156,158, which can be formed integrally with or otherwise attached to the core wire 152. A number of collars 160,162,164,166 coupled to the filter struts 136 allow the filter basket 126 to slide and rotate on the pusher wire 128.

The basket cells 140 forming the filter basket 126 can be configured to expand between a collapsed position and an expanded position within the body. To retrieve a blood clot within a blood vessel, the retrieval device 124 can be loaded into the inner lumen of a delivery device in its unexpanded state, inserted into the patient's body, and then advanced through the vasculature to a target site using the pusher wire 126. Once positioned at or near the blood clot, the retrieval device 124 can then be withdrawn from the delivery device, causing the filter basket 124 to radially expand within the blood vessel.

Once withdrawn from the delivery device, the physician may next pull the core wire 152 proximally while holding the pusher wire 128 stationary within the blood vessel, causing the filter basket 126 to move proximally along the pusher wire 128. A proximal stop 168 attached to the pusher wire 128 can be configured to limit proximal movement of the filter basket 126 along the pusher wire 128. Once in contact with the proximal stop 168, continued pulling of the core wire 152 in the proximal direction causes the proximal-most collar 160 to compress against the proximal stop 168, which, in turn, compresses the filter basket 126 axially along its length. When compressed in this manner, the basket cells 140 of the filter basket 126 radially expand within the blood vessel. To vary the size that the expanded filter basket 126 assumes within the blood vessel, the physician may vary the proximal force exerted on the core wire 152, as desired.

FIG. 13 is a top view of the filter basket 124 of FIG. 12, showing the filter basket 126 prior to assembly on the pusher wire. As shown in FIG. 13, the filter basket 126 may have a unitary construction formed from a single unitary workpiece such as a flat sheet or a tubular structure. In some fabrication methods, a laser machining, laser etching, chemical etching, or photochemical etching process can be used to cut the workpiece to form the various elements of the device. The filter basket 126 can then be attached to the collars 160,162,164,166 (see FIG. 12) using a suitable bonding technique such as soldering, crimping, brazing, adhesion, etc. In some embodiments, all or a portion of the filter basket 126 may have a textured surface thereon formed, for example, by applying a nanoporous coating to all or selective portions of the filter struts 136. Other features such as radiopaque markers can also be placed on selective filter struts 136 to enhance radiographic visualization of the device within the body.

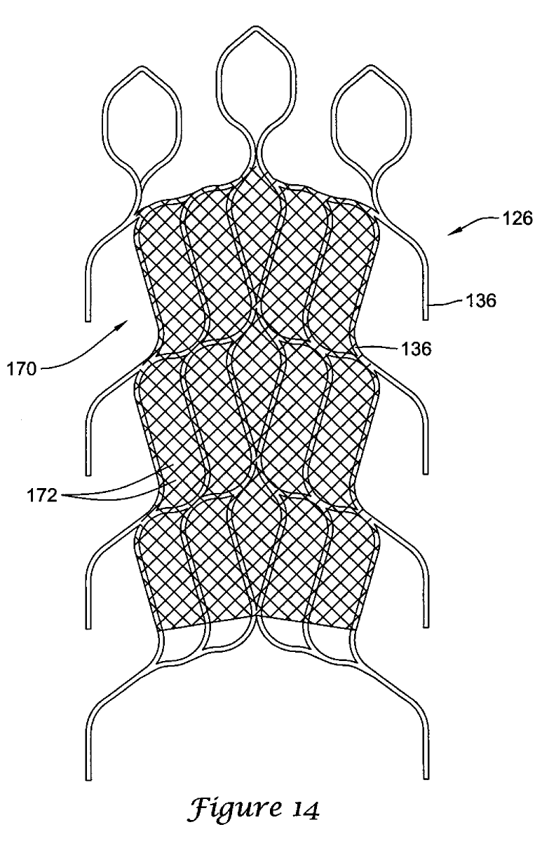

The filter basket 126 may further include a polymeric web covering to further capture the blood clot or any other emboli therein. As shown in FIG. 14, for example, a polymeric web 170 can be coupled to selective filter struts 142 on the filter basket 126. The polymeric web 170 can include a number of openings or pores 172 of sufficient size to capture the blood clot and any emboli while maintaining the perfusion of blood through the filter basket 126.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. Changes may be made in details, particular in matters of size, shape, and arrangement of parts without exceeding the scope of the invention. It will be understood that this disclosure is, in many respects, only illustrative.

What is claimed is:

1. A retrieval device for removing a foreign object from a body lumen, comprising:
a flexible coil movable between a first position and a second position within a body lumen, the flexible coil adapted to assume a substantially straight shape in the first position and an expanded shape forming one or more loops in the second position; and
a core wire operatively coupled to the flexible coil, the core wire having a proximal section and a distal section;
wherein the core wire is located externally to the flexible coil.

2. The retrieval device of claim 1, wherein the flexible coil comprises a coiled flat ribbon.

3. The retrieval device of claim 1, wherein the flexible coil includes a superelastic material.

4. The retrieval device of claim 1, wherein the flexible coil includes a shape memory material.

5. The retrieval device of claim 1, wherein the flexible coil has a textured surface.

6. The retrieval device of claim 5, wherein the flexible coil includes a number of bumps or protrusions.

7. The retrieval device of claim 5, wherein said textured surface includes a nanoporous coating.

8. The retrieval device of claim 1, wherein said one or more loops are adapted to align circumferentially with the inner wall of the body lumen.

9. The retrieval device of claim 1, wherein said one or more loops comprise a single loop.

10. The retrieval device of claim 1, wherein said one or more loops comprise a plurality of loops.

11. The retrieval device of claim 10, wherein said plurality of loops have a distally tapering shape.

12. The retrieval device of claim 1, further comprising one or more fibers operatively coupled to the flexible coil, each fiber adapted to constrain longitudinal movement of the flexible coil in the second position.

13. The retrieval device of claim 12, wherein at least one of the fibers forms a proximal mouth of the flexible coil.

14. The retrieval device of claim 1, further comprising an actuator mechanism operatively coupled to the proximal section of the core wire.

15. The retrieval device of claim 1, wherein the flexible coil is predefined to be in the expanded shape.

16. The retrieval device of claim 1, wherein the flexible coil is movable between the first position and the second position in response to an axial force applied to the core wire.

17. The retrieval device of claim 1, wherein the one or more loops are configured in a substantially helical orientation.

18. The retrieval device of claim 1, wherein the flexible coil defines a first path and the core wire defines a second path;
wherein the first path is substantially different from the second path when the flexible coil is in the expanded shape.

19. The retrieval device of claim 1, wherein the flexible coil defines a first path and the core wire defines a second path;
wherein the first path is substantially parallel to the second path when the flexible coil is in the substantially straight shape.

20. A retrieval device for removing a foreign object from a body lumen, comprising:
an elongated member including a flexible coil movable between a first position and a second position within a body lumen, the flexible coil adapted to assume a substantially straight shape in the first position and an expanded shape forming a plurality of helically oriented loops in the second position;
a core wire operatively coupled to the flexible coil, the core wire having a proximal section and a distal section;
wherein the core wire is located externally to the flexible coil;
one or more fibers each operatively coupled at a plurality of locations to the flexible coil, each fiber adapted to constrain longitudinal movement of the flexible coil in the second position; and
an actuator mechanism operatively coupled to the proximal section of the core wire.

21. The retrieval device of claim 20, wherein the flexible coil includes a coiled flat ribbon.

22. The retrieval device of claim 20, wherein the flexible coil includes a shape memory material.

23. The retrieval device of claim 20, wherein the flexible coil includes a superelastic material.

24. The retrieval device of claim 20, wherein the flexible coil is predefined to be in the expanded shape.

25. The retrieval device of claim 20, wherein the flexible coil is movable between the first position and the second position in response to an axial force applied to the core wire.

26. The retrieval device of claim 20, wherein the flexible coil defines a first path and the core wire defines a second path;
wherein the first path is substantially different from the second path when the flexible coil is in the expanded shape.

27. The retrieval device of claim 20, wherein the flexible coil defines a first path and the core wire defines a second path;
wherein the first path is substantially parallel to the second path when the flexible coil is in the substantially straight shape.

28. The retrieval device of claim 20, wherein the flexible coil has a textured surface.

29. The retrieval device of claim 28, wherein the flexible coil includes a number of bumps or protrusions.

30. The retrieval device of claim 28, wherein the textured surface includes a nanoporous coating.

31. The retrieval device of claim 20, wherein at least one of the fibers forms a proximal mouth of the flexible coil section.

32. The retrieval device of claim 20, wherein said plurality of loops have a distally tapering shape.

33. A method for removing a foreign object from a body lumen comprising the steps of:
providing a retrieval device having an elongated member including a flexible coil movable between a first collapsed position and a second expanded position in response to an axial force applied to a core wire operatively coupled to the flexible coil, where the retrieval device further includes one or more fibers adapted to limit longitudinal movement of the flexible coil operatively coupled to the flexible coil;
introducing the retrieval device into a body lumen with the flexible coil in the collapsed position;
applying an axial force to the core wire to move the flexible coil into the expanded position;
capturing a foreign object within the body lumen; and
removing the retrieval device and captured foreign object from the body lumen.

34. The method of step 33, wherein the retrieval device is further provided with an actuator mechanism operatively coupled to the core wire.

35. The method of step 34, wherein the axial force is applied using said actuator mechanism.
